(19) United States
(12) Patent Application Publication
(10) Pub. No.: US 2023/0220122 A1
(43) Pub. Date: Jul. 13, 2023

(54) METHOD FOR PRODUCTION OF FUNCTIONAL POLYSACCHARIDE AND COSMETIC COMPOSITION

(71) Applicant: CARBOEXPERT INC., Daejeon (KR)

(72) Inventors: Chang Kyu LEE, Daejeon (KR); Min Jee YOO, Sejong (KR); Hyun Ah YU, Daejeon (KR); Jin Ju BAE, Daejeon (KR)

(73) Assignee: CARBOEXPERT INC., Daejeon (KR)

(21) Appl. No.: 17/436,807

(22) PCT Filed: Sep. 18, 2019
(86) PCT No.: PCT/KR2019/012103

§ 371 Date: Apr. 24, 2022

(51) Int. Cl. C08B 37/00; A61K 8/73; A61Q 19/00; A61Q 19/08; C12N 1/20

(52) U.S. Cl. CPC C08B 37/0006; A61K 8/73; A61Q 19/007; A61Q 19/08; C12N 1/205; C12R 2001/19

(57) ABSTRACT

Provided are a method of manufacturing a functional polysaccharide, the method including: preparing a cell containing a polysaccharide; crushing the cell to extract polysaccharide to the outside of the cell to obtain an extract; and purifying the extract by removing at least one of crude protein, crude fat, and crude powder from the extract, and a cosmetic composition for skin moisturizing or skin elasticity improvement, the cosmetic composition including the functional polysaccharide as an active ingredient.

### TECHNICAL FIELD

The present disclosure relates to a method of producing a functional polysaccharide and a cosmetic composition.

### BACKGROUND ART

Polysaccharides produced to date are usually extracted from plants such as barley, ginseng, mugwort, ginkgo, chlorella and some seaweed, and most of the polysaccharides currently used are extracted from edible or medicinal mushrooms and commercialized. The method is obtained by hot-water extracting fruit bodies or mycelium. However, this technical method has limitations in the supply and demand of raw materials due to limited production of the fruit bodies, and in the case of extraction from the mycelium, polysaccharides cannot be obtained in a large amount. Moreover, in the case of some mushrooms that are not artificially cultured, it is almost impossible to mass-produce polysaccharides from fruit bodies.

Glycogen, one of the polysaccharides, is one of the storage polysaccharides that exist widely in the body of animals. Glycogen exists in the liver or muscle and exists as granules that are insoluble in water in the cytoplasm. Glycogen occupies 2 wt % to 10 wt % in the liver, 1 wt % to 2 wt % in the muscle. Glycogen in the liver or the muscle varies depending on the glucose value (blood sugar level) in the blood or the amount of exercise. When the blood sugar level rises, the synthesis of glycogen is promoted, and when the blood sugar level decreases, the decomposition of glycogen is accelerated. Accordingly, the physiological significance thereof is high because the blood sugar level is maintained constant. In the liver, in glucose-6-phosphate (G-6-P), due to the action of enzyme phosphatase, the phosphate group is separated therefrom and glucose is released into the blood. Muscles do not have this phosphatase.

Therefore, muscle glycogen does not contribute to the increase in blood sugar level. In muscles, glycogen is decomposed by exercise to produce G-6-P, but glycogen is only used in muscle cells.

Two control mechanisms are known for the synthesis or degradation of glycogen: a control by hormones; and a control by materials that act as enzymes involved in the synthesis or breakdown of glycogen. As such hormones, insulin or cortical corticoids act on the synthesis of glycogen, and adrenaline and glucagon act on the decomposition of glycogen. As such materials, Ca2+ or cAMP (cyclic adenosine-3′, 5′-monophosphate, cyclic AMP) may be used, and these materials change the structure of enzymes involved in the synthesis or decomposition of glycogen. This is called an allosteric control mechanism.

In the structure of glycogen, glucose molecules are bonded in the shape of branches. In this aspect, glycogen is also called plant starch. The molecular weight thereof is around one million. As for the structure, to 10 consecutive glucose molecules bound by α-1,4 glycoside bond, other glucose molecules are bound by α-1,6 glycoside bond, to which glucose molecules are bound by α-1,4 glycoside bond. As such, glycogen has the shape of a twig. This structure is similar to amylopectin, but glycogen has greater number of branches and shorter branches. In the iodine reaction, starch appears purple or reddish-purple, while glycogen appears red or brown.

Meanwhile, Korean Patent Publication No. 2010-0095829 discloses ‘a mutant of Saccharomyces cerevisiae producing a high concentration of glutathione and a method of mass-producing glutathione by culturing the same.’ Korean Patent No. 700910 discloses ‘a yeast mutant that produces an alkali-soluble β-glucan.’

As one of the methods to increase productivity, the production technology of polysaccharides using cell culture methods is being studied. However, in obtaining polysaccharides from cells, the purity of the polysaccharides is decreased and the activity is decreased, and the toxicity of cells and skin due to unwanted ingredients may occur.

### DESCRIPTION OF EMBODIMENTS

### Technical Problem

Embodiments of the present disclosure provide a method of producing a functional polysaccharide with high concentration and high purity.

Embodiments of the present disclosure provide a cosmetic composition for skin moisturizing or skin elasticity improvement, using a high purity of functional polysaccharide as an active ingredient.

### Solution to Problem

The present disclosure provides a method of manufacturing a functional polysaccharide, the method including: preparing a cell containing a polysaccharide; crushing the cell to extract polysaccharide to the outside of the cell to obtain an extract; and purifying the extract by removing at least one of crude protein, crude fat, and crude powder from the extract.

In an embodiment, in the preparing the cell containing the polysaccharide, an E. coli mutant TBP38 strain that high-produces polysaccharide, is cultured to prepare a cell.

In an embodiment, in the crushing the cell to extract the functional polysaccharide to the outside of the cell to obtain an extract, the cell is suspended in a citric acid buffer solution having a pH of 3.5 to 4.5, and then the cell is crushed by high pressure treatment to extract the functional polysaccharide to the outside of the cell.

In addition, in the purifying of the extract, heat-treating the extract at 80 to 100° C. for 5 minutes to 60 minutes; adding citric acid to adjust the pH to be 3.5 to 4.5 and remove the precipitate therefrom; separating layers by adding a detergent and recovering a supernatant through centrifugation; filtering the recovered supernatant through an ultrafine filter and concentrating the same to obtain a concentrate; adding alcohol in an amount 1 to 5 times the volume of the concentrate to recover the precipitate; and re-suspending the precipitate in distilled water or deionized water, and then re-adding alcohol in an amount 1 to 5 times the volume of the precipitate and recovering the precipitate.

In an embodiment, an amount of each of crude protein, crude fat, and crude powder is 0.5% or less.

In an embodiment, the polysaccharide may be glycogen, and may have a purity of 99% or more based on a solid content.

Embodiments of the present disclosure provide a functional polysaccharide in which the amount of each of crude protein, crude fat, and crude powder is 0.5% or less, and 99% or more of the functional polysaccharide is glycogen based on solid content.

In an embodiment, the functional polysaccharide has antioxidant activity.

In an embodiment, at a concentration of 1000 μg/ml or less, the functional polysaccharide does not have phototoxicity to the BALB/3T3 clone A31 cell line.

Embodiments of the present disclosure provide a cosmetic composition for skin moisturizing or skin elasticity improvement, including the functional polysaccharide as an active ingredient.

### Advantageous Effects of Disclosure

The present disclosure provides a method of producing a functional polysaccharide capable of producing a polysaccharide with a high purity of 99% or more.

The present disclosure also provides a functional polysaccharide having an antioxidant activity, a skin moisturizing function, a skin elasticity improvement function and having no or significantly low cytotoxicity, skin toxicity, phototoxicity, and a cosmetic composition including the same.

The above effects and additional effects will be described in detail below.

### BRIEF DESCRIPTION OF DRAWING

FIG. 1 is a flowchart illustrating a method of producing a functional polysaccharide according to an embodiment of the present disclosure.

FIG. 2a is a graph illustrating a result of cell viability of a test substance when UV was not irradiated.

FIG. 2b is a graph illustrating a result of cell viability of a test substance when UV was irradiated.

FIG. 2c is a graph illustrating a result of cell viability of a positive control when UV was not irradiated.

FIG. 2d is a graph illustrating a result of cell viability of a positive control when UV was irradiated.

FIG. 3a is a graph illustrating a result of cytotoxicity of a test substance.

FIG. 3b is a graph illustrating a result of cytotoxicity of a positive control.

FIG. 4 is a graph illustrating a result of cytotoxicity according to dilution concentration.

FIG. 5 is a graph illustrating age distribution of test subjects.

FIG. 6a is a graph illustrating improvement of moisture content in a test group.

FIG. 6b is a graph illustrating improvement rate of moisture content in a test group.

FIG. 7a is a graph illustrating improvement of elasticity in a test group.

FIG. 7b is a graph illustrating improvement rate of elasticity in a test group.

### MODE OF DISCLOSURE

Before describing the present disclosure in detail below, it shall be understood that the terms used in the present specification are presented herein for describing specific embodiments, and are not intended to limit the scope of the present disclosure, which is limited only by the scope of the appended claims. All technical and scientific terms used in this specification have the same meaning as commonly understood by those of ordinary skill in the art unless otherwise stated.

Throughout this specification and claims, unless otherwise stated, the terms (comprise, comprises, comprising) include the described object or step, or a group of objects or steps, and may not exclude any other object or step, or a group of other objects or steps.

On the other hand, various embodiments of the present disclosure may be combined with any other embodiments unless described otherwise. Hereinafter, embodiments of the present disclosure and effects thereof will be described.

A method of producing a functional polysaccharide according to an embodiment of the present disclosure includes: preparing a cell containing a polysaccharide; crushing the cell to extract the polysaccharide to the outside of the cell to obtain an extract; and purifying the extract by removing at least one of crude protein, crude fat, and crude powder from the extract.

In the preparing the cell containing the polysaccharide, an E. coli mutant TBP38 strain that high-produces polysaccharide, is cultured to prepare a cell. In addition, as a strain that is used in addition to the above strain, a Saccharomyces cerevisiae CEY1 mutant strain that has the accession number of KCTC18748P and high-produces glycogen, a Saccharomyces boulardii CEY2 mutant strain that has the accession number of KCTC18749P, and the like may be used.

The culture method and the culture medium used are not limited. For example, a multi-stage culture method may be used. In the first culture, a medium containing glucose as the primary carbon source may be used, and a medium containing 1-3 g/L (NH₄)₂·HPO₄, 6-9 g/L KH₂PO₄, 0.5-1.5 g/L citric acid, 0.5-1 g/L MgSO₄·7H₂O, 15-25 g/L glucose, and a trace amount of metal solution may be used, but the medium for the first culture is not limited thereto. In the second culture, after the first culture, a secondary carbon source is added and then 20 to 30 hours of culture is performed to obtain a cell. The secondary carbon source may be selected from glucose, maltose, maltotriose, maltotetraose, maltopentaose, and maltohexaose, but is limited thereto. The secondary carbon source may be corn starch saccharified liquid powder, but is not limited thereto.

In the crushing the cell to extract the functional polysaccharide to the outside of the cell to obtain an extract, the cell is suspended in a citric acid buffer solution having a pH of 3.5 to 4.5, and then the cell is crushed by high pressure treatment to extract the functional polysaccharide to the outside of the cell. In an embodiment, the pH may be from 3.9 to 4.1. In one or more embodiments, cells may be crushed by ultrasonic treatment instead of high-pressure treatment, and the present disclosure is not limited thereto. The high-pressure treatment may be performed by two consecutive high-pressure treatments at 1200 psi using a homogenizer, so as to crush cells.

Next, the purification process is performed to obtain a functional polysaccharide with high purity.

First, the extract may be heat-treated at 80° C. to 100° C. for 5 minutes to 60 minutes. Thereafter, a citric acid is added to adjust the pH to be 3.5 to 4.5, or 3.9 to 4.1. As such, proteins and cell debris precipitate, and the supernatant thereof is recovered by using a semi-continuous tubular type centrifuge to remove primary impurities therefrom.

In an embodiment, a detergent may be added to the recovered supernatant, to remove endotoxin. An example thereof is Triton X-114, but is not limited thereto. The detergent is added at a concentration of 0.1% (v/v) to 5% (v/v) and mixed, and then allowed to stand still at 30° C. to 40° C. sufficiently to cause layer separation, followed by centrifugation (12000 rpm, 30° C., 15 min) to recover a supernatant. Subsequently, if necessary, the detergent treatment may be further performed multiple times, for example, twice, to recover the supernatant. By doing so, impurities may be further removed.

Thereafter, the recovered supernatant may be filtered through an ultra-fine filter to remove fine-sized proteins and nucleic acids such as DNA. The ultra-fine filter is not limited. In an embodiment, an ultra-fine filter having 300 kDa size membrane or more may be used. Once the filtration is completed, a concentrating process may be performed.

Then, alcohol in an amount 1 to 5 times the volume (v/v) of the concentrate may be added to the concentrate, followed by centrifugation (12000 rpm, 4° C., 15 min) to recover a precipitate (containing functional polysaccharide). In order to obtain higher purity, the precipitate may be re-suspended in distilled water, and then alcohol in an amount 1 to 5 times the volume (v/v) of the precipitate is re-added, followed by centrifuging to recover the precipitate. The precipitate may be freeze-dried to finally obtain a high-purity functional polysaccharide.

In the functional polysaccharide obtained by the processes described above, each of crude protein, crude fat, and crude powder may have an amount 0.5% or less by weight, and, for example, an amount of crude protein may be 0.50% or less, an amount of crude fat may be 0.1% or less, and an amount of crude may be 0.45% or less. In this case, the functional polysaccharide may be obtained with very high purity. The functional polysaccharide may be dominated by glycogen, and the functional polysaccharide may have a purity of 99% or more based on a solid content.

On the other hand, as shown in the embodiments to be described later, the high-purity functional polysaccharide according to the present disclosure has antioxidant activity, does not have phototoxicity to BALB/3T3 clone A31 cell lines at a concentration of 1000 μg/ml or less, and has remarkably low skin toxicity and cytotoxicity.

A cosmetic composition including the functional polysaccharide according to the present disclosure as an active ingredient is very excellent in skin moisturizing and skin elasticity improvement performance.

The formulation of the cosmetic composition is not limited, and any known formulation may be used.

Skin and body care cosmetics containing the cosmetic composition may be prepared in various formulations, such as essences, ampoules, soaps, tonics and body essences, emulsions, lotions, creams (oil-in-water type, water-in-oil type, multi-phase), solutions, suspensions (anhydrous and water-based), anhydrous products (oil and glycol-based), gels, powders, etc., and the cosmetic composition may be contained in an amount of 0.05 wt % to 100 wt % based on the total cosmetics, and in the case of an ampoule formulation, an amount of the functional composition may be 100%.

In addition to the functional polysaccharide, the cosmetic composition may include a carrier acceptable in a skin cosmetic preparation. Examples of the carrier include alcohols, oils, surfactants, fatty acids, silicone oils, preservatives, wetting agents, moisturizing agents, viscosity modifiers, emulsions, stabilizers, sun-screening agents, coloring agents, fragrances, and diluents.

Specific compounds or compositions that can be used as the alcohols, oils, surfactants, fatty acids, silicone oils, preservatives, wetting agents, moisturizing agents, viscosity modifiers, emulsions, stabilizers, sun-screening agents, coloring agents, fragrances, diluents, etc. are already known in the art. Therefore, those skilled in the art can select and use an appropriate compound or composition.

Examples of alcohols include water-soluble polyhydric alcohols such as higher alcohol, propylene glycol, 1,3-butylene glycol, glycerin, sorbitol, and polyethylene glycol, examples of oils include avogade oil, palm oil, beef tallow, and Jojoba oil, examples of preservatives include ethylparaben ad butylparaben, examples of moisturizing agents include hyaluronic acid, chondroitin sulfate, and pyrrolidone carboxylate, and examples of diluents are ethanol and isopropanol.

In an embodiment, when the formulation of a functional cosmetic is a paste, cream or gel, animal fibers, plant fibers, wax, paraffin, starch, tracant, cellulose derivatives, polyethylene glycol, silicone, bentonite, silica, talc, or zinc oxide may be used as a carrier component.

In an embodiment, when the formulation is a powder or spray, lactose, talc, silica, aluminum hydroxide, calcium silicate, or polyamide, each in the form of powder, may be used as a carrier component. In the case of a spray, a propellant, such as chlorofluorohydrocarbon, propane/butane, or dimethyl ether, may be used.

In addition, when the formulation is a solution or an emulsion, a solvent, a solvating agent, or an emulsifying agent may be used as a carrier component. Examples thereof are water, ethanol, isopropanol, ethyl carbonate, ethyl acetate, benzyl alcohol, benzyl benzoate, propylene glycol, 1,3-butyl glycol oil, glycerol aliphatic ester, polyethylene glycol, or fatty acid ester of sorbitan.

In an embodiment, when the formulation is a suspension, liquid diluents such as water, ethanol or propylene glycol, ethoxylated isostearyl alcohol, suspending agents such as polyoxyethylene sorbitol ester and polyoxyethylene sorbitan ester, microcrystalline cellulose, aluminum metahydroxide, bentonite, agar, or tracant, may be used as a carrier component.

In addition, when the formulation is a surfactant-containing cleansing, aliphatic alcohol sulfate, aliphatic alcohol ether sulfate, monoester sulfosuccinate, isethionate, imidazolinium derivative, methyltaurate, sarcosinate, fatty acid amide ether sulfate, alkylamidobetaine, fatty alcohol, fatty acid glyceride, fatty acid diethanolamide, vegetable oil, a linoline derivative, or ethoxylated glycerol fatty acid ester, may be used as a carrier component.

In an embodiment, in the case of a soap containing the functional polysaccharide according to an embodiment of the present disclosure, the soap may be produced by including a functional polysaccharide in a soap base, and, as an additive, a skin moisturizer, an emulsifier, a water softener, and the like may be included.

Examples of the soap base include vegetable oils such as coconut oil, palm oil, soybean oil, caster oil, olive oil, and palm kernel oils, or animal oils such as beef tallow, pork fat, brisket, and fish oil, examples of the skin moisturizer include glycerin, erythritol, polyethylene glycol, propylene glycol, butylene glycol, pentylene glycol, hexyl glycol, isopropyl myristate, silicone derivatives, aloe vera, and sorbitol, examples of the emulsifier include natural oils, wax fatty alcohols, hydrocarbons, and natural plant extracts, and examples of the water softeners include tetrasodium EDTA.

The soap composition according to the present disclosure may further include, as an additive, an antimicrobial agent, a dispersant, a foam inhibitor, a solvent, a scale inhibitor, a corrosion inhibitor, a fragrance, a colorant, a metal ion sequestering agent, an antioxidant, a preservative, and the like.

Hereinafter, a method of preparing a functional polysaccharide and a cosmetic composition according to an embodiment of the present disclosure will be described in detail through examples. The following examples are described herein for illustrative purpose only, and the scope of the present disclosure is not limited to the following examples.

### <Example 1> Preparation of Functional Polysaccharide Extraction of Functional Polysaccharide

A secondary carbon source was added to the culture medium in which Escherichia coli mutant TBP38 strain, which high-produces functional polysaccharide, had been cultured in a medium containing glucose as a primary carbon source, and then, the strain was cultured for 20 to 30 hours to obtain cultured cells.

In order to extract polysaccharide from the cells obtained through cell culture at a high concentration, first, the cells obtained through centrifugation were resuspended in a pH 4.0 citric acid buffer solution. Thereafter, the cells were crushed by continuous high-pressure treatment at 1200 psi twice using a homogenizer, and then the functional polysaccharide was extracted to the outside of the cells to obtain an extract.

Purification of Functional Polysaccharide

In order to perform the purification process by removing the crude protein, crude fat, and crude powder contained in the functional polysaccharide extract, first, a heat-treatment process was performed at 90° C. for 20 minutes, and then citric acid was added thereto to adjust the pH of the extract to be 4.0. Then, the supernatant was recovered using a semi-continuous tubular type centrifuge to remove precipitated proteins and cell debris. To remove endotoxin, Triton X-114, which is a detergent, was added at a concentration of 1% (v/v) to the recovered supernatant, and then, left the same sufficiently at 37° C. to cause layer separation, followed by centrifugation (12000 rpm, 30° C., 15 min). The recovered supernatant was treated twice with Triton X-114 having a concentration of 1% (v/v), followed by centrifuging to obtain the supernatant. That is, in this experiment, Triton X-114 was performed three times. Using ultra filtration (300 kDa size membrane), small-sized proteins and nucleic acids such and DNA remaining in the recovered supernatant were filtered off and concentrated. Then, alcohol in an amount 2 times the volume (v/v) of the concentrate was added to the concentrate, followed by centrifugation (12000 rpm, 4° C., 15 min) to recover a precipitate (containing functional polysaccharide). Thereafter, the precipitate was resuspended in distilled water, and then alcohol in an amount 2 times the volume (v/v) of the precipitate was re-added, followed by centrifuging to recover the precipitate, which was then freeze-dried to obtain a functional polysaccharide.

### Example 2

A functional polysaccharide was obtained in the same manner as in Example 1, except that Saccharomyces cerevisiae CEY1 mutant strain was used instead of Escherichia coli variant TBP38 strain.

### Comparative Example 1

A functional polysaccharide was obtained in the same manner as in Example 1, except that, in the purifying process, the obtained cultured cells were subjected to high-temperature and high-pressure treatment for 20 minutes to 60 minutes at a temperature of 110° C. to 130° C. at 10 to 30 psi, and then ethanol was added to precipitate the polysaccharide, and the polysaccharide was filtered from the precipitate to obtain a functional polysaccharide.

### <Example 3> Purity Measurement

### <Example 3-1> Moisture Content Measurement (Heating Loss Method)

Devices used herein were a drying oven, a desiccator, an aluminum crucible, and a balance.

A sample in an amount of 2 g to 5 g, which was accurately measured using an aluminum crucible which had been dried and of which constant weight had been measured, was dried at a temperature of 135±2° C. exactly for 2 hours or dried until reaches a constant weight at a temperature of 105° C. to 110° C., by using a constant temperature dryer, and allowed to cool in a desiccator for 30 minutes. Then, the weight thereof was measured and the weight loss was determined as the moisture content. The moisture content was calculated and expressed as the following equation.

$$Moisture content ( \% ) = \frac{\begin{matrix}weight before dry ( crucible + s ample ) - \\ weight after dry ( crucible + s a m p l e )\end{matrix}}{w eight of sample} \times 100$$

### <Example 3-2> Measurement of Crude Protein Content (Automatic Analysis Method, Kieltec Method)

Devices and reagents used herein were a digestion apparatus, a sample injection and distillation apparatus, a chemical balance, a magnetic stirrer, a volumetric flask, a measuring cylinder, a decomposition bottle, a decomposition bottle stand, a reagent dispenser, and sulfuric acid (reagent class 1).

To prepare a reagent, 87 ml of 1 N hydrochloric acid: concentrated hydrochloric acid (specific gravity 1.18) was diluted with distilled water to 10 liters, and the factor was calculated as follows.

$$Factor ( F ) = 188.67 \times \frac{X}{b}$$

$$x : weight of sodium carbonate ( g )$$

$$b : amount of 0.1 N HCL used for filtration$$

As a method of determining the factor, standard sodium carbonate was heated at 260° C. to 270° C. for about 1 hour, and then allowed to cool in a desiccator for 30 minutes, and a portion of the result was accurately weighed until the weight of the portion reached 1.5 g. The portion was dissolved and mixed in distilled water in such an amount that the solution reached the mark of a 250 ml volumetric flask. Then, 25 ml thereof was taken therefrom and two drops of methyl orange was slowly dropped thereto. Then, 0.1 N hydrochloric acid was slowly dropped thereto, and when the resultant solution turned from yellow into pink, the solution was boiled for 2 minutes to 3 minutes. After cooling, the titration was performed again until the resultant solution turns into pink again. At this time, the factor of 0.1 N hydrochloric acid solution was calculated from the consumed mW of 0.1 N hydrochloric acid solution.

Decomposition accelerator: prepared by mixing potassium sulfate (K₂SO₄) and copper sulfate (CuSO₄·5H₂SO) at a ratio of 10 g:1 g thoroughly in a mortar.

40% sodium hydroxide solution: prepared by dissolving 4 kg of sodium hydroxide (NaOH) in distilled water in such an amount that the amount of the resultant solution reached 10 [symbol] .

1% boric acid solution: 100 g of boric acid (H₃BO₃) was dissolved in distilled water to obtain 10 f of the solution, and 100 ml of a bromocresol green solution and 70 ml of a methyl red solution (100 mg of bromocresol green and 100 mg of methyl red each dissolved in 100 ml of methanol) were each added thereto.

Preparation of Sample Solution (Decomposition)

0.7 g to 1 g of samples of Example 1, Example 2 and Comparative Example 1 were pored into a decomposition bottle, and 7 g to 8 g of a decomposition accelerator was added thereto and mixed well together, and then 10 ml of sulfuric acid (H₂SO₄) was slowly added and mixed. At first, the resultant solution was gradually heated so that the bubbles did not overflow, and when bubbles did not occur, the solution was strongly heated until it became transparent to decompose. After that, the crude protein was measured according to the operating method of the automatic analyzer.

### <Example 3-3> Measurement of Crude Fat Content (Ether Extraction Method)

Devices used herein were a fat extraction device (FOSS, Soxtec 1043), a dryer, a desiccator, No. 2 filter paper, ethyl ether, etc.

A fat measuring bottle was dried at 95° C. to 100° C. for about 2 hours, allowed to cool in a desiccator for 30 minutes, and weighed. 1 g to 2 g of a sample was placed in a cylindrical filter, covered with cotton wool, and dried at 95° C. to 100° C. for about 2 hours. After that, the sample was placed in a fat extraction device, and ethyl ether was pored thereinto and heated at a temperature of 110° C. to extract fat for 1 hour, and the ethyl ether was recovered therefrom. The fat measuring bottle was dried at a temperature of 95° C. to 100° C. for 3 hours, and then allowed to cool in a desiccator for 30 minutes and the weight thereof was measured. A weight obtained by subtracting the weight of the fat measuring bottle therefrom, was calculated as a percentage point with respect to the amount of the sample. The result was considered as the crude fat content.

The crude fat content was calculated through the following equation.

$$Crude fat ( \% ) = \frac{\begin{matrix}fat crucible weight after extract - \\ fat crucible weight before extract\end{matrix}}{weight of sample} \times 100$$

### <Example 3-4> Measurement of Crude Powder Content

Devices used herein were an electric muffle furnace (maintained at 600° C.), a porcelain crucible, a desiccator with silica gel desiccant, a metal tong, a balance, etc.

The clearly washed crucible was left for 1 hour to 2 hours in a muffle furnace at 600° C., and then, transferred to a desiccator and allowed to cool to room temperature (40 minutes), and the weight thereof was measured. 1 g to 2 g of a sample was taken and placed in a crucible, preliminarily incinerated by heating with an electric stove or gas burner, and then incinerated for 2 hours in a muffle furnace at 600° C. At this time, the color of the ash was made to be white, light gray, or reddish. Then, the weight of the ash was measured after cooling for 40 minutes in a desiccator.

The crude powder content was calculated through the following equation.

$$Crude powder ( \% ) = \frac{w eight of powder}{w eight of sample} \times 100$$

TABLE 1

| | | | Comparative |
|---|---|---|---|
| Test item | Example 1 | Example 2 | Example 1 |
| Moisture | 5.39% | 5.54% | 5.00% |
| Crude protein | 0.50% | 0.50% | 16.58% |
| Crude fat | 0.02% | 0.07% | 0.19% |
| Crude powder | 0.24% | 0.41% | 5.81% |

As shown in the table above, in Example 1 and Example 2, compared to Comparative Example 1, the amounts of crude protein, crude fat, and crude powder were all 0.50% or less, indicating that the samples were purified well. In the case of the polysaccharide which was purified after moisture was removed therefrom, the purity thereof was confirmed to be more than 99%.

### <Example 4> Antioxidant Effect

To measure the antioxidant effect of functional polysaccharides, DPPH assay and ABTS assay were performed to measure radical scavenging ability.

### <Example 4-1> DPPH Assay

After injecting, into a 96 well plate, 0.1 ml of each of a sample and DPPH solution in a 1:1 ratio, the reaction was caused for 30 minutes in a dark place at 35° C., and then the absorbance was measured at 517 nm.

The radical scavenging ability (%) was calculated as follows.

$$Radical scanvenging ability ( \% ) = 1 - \frac{a b s o r bance of sample}{a b s o r bance of negative control} \times 100$$

The sample used in this experiment was used after manufacturing 1000 g/20 L at a concentration of 5%.

DPPH solution: 0.078 g of DPPH and 94% ethanol were used to prepare a 1 L solution, and the solution was used after the concentration thereof reached 0.2 mM.

Sample use: 94% ethanol was used to dilute 10 times.

Negative control: 100 mM DPPH solution in 94% ethanol (94% ethanol+200 mM DPPH solution) was used.

The results are shown in Table 2.

TABLE 2

| | | | Comparative |
|---|---|---|---|
| | Example 1 | Example 2 | Example 1 |
| 1 | 29.84 | 28.55 | 18.54 |
| 2 | 28.99 | 28.15 | 19.47 |
| 3 | 29.88 | 28.52 | 20.14 |
| Ave | 29.12 | 28.41 | 19.38 |
| SD | 0.217 | 0.223 | 0.80 |

Radical scavenging ability measurement result (unit: %)

As a result of the DPPH assay, Examples 1 and 2 respectively showed, at 5% concentration, the average radical scavenging activity of 28.41% and 29.12%, which are statistically significant values compared to the negative control.

### <Example 4-2> ABTS Assay

10 μl of each of a sample and trolox standard was added to a 96 well plate, and then, 20 μl of myoglobin working solution and 150 μl of ABTS substrate working solution were added thereto and reacted at room temperature for 5 minutes. After the reaction was finished, 100 μl of a stop solution was injected and an absorbance was measured at 405 nm.

The calculation method of the radical scavenging ability (%) was the same as the DPPH assay.

Myoglobin Stock Solution: 280 μl of ultrapure water was added to myoglobin from horse heart.

Myoglobin Working Solution: myoglobin stock solution was diluted 100 times using 1×assay buffer.

Trolox Working Solution: 2.67 ml of 1×assay buffer was added to trolox ((±)-6-hydroxy-2,5,7,8-tetramethylchromane-2-carboxylic acid.

ABTS Working Solution: Prepared by adding 25 μl of 3% hydrogen peroxide solution to 10 ml of ABTS substrate solution.

Sample use: Used after diluting 10 times using 1×ABTS assay buffer.

Negative control: ABTS mixture solution (1×ABTS assay buffer+myoglobin working solution+ABTS substrate working solution) was used.

| | 1.5 mM Trolox | | Trolox |
|---|---|---|---|
| | Working Solution | 1× assay buffer | concentration in |
| Tube | (ul) | (ul) | the standard (mM) |
| 1 | 0 | 500 | 0 |
| 2 | 5 | 495 | 0.015 |
| 3 | 15 | 485 | 0.045 |
| 4 | 35 | 165 | 0.105 |
| 5 | 70 | 430 | 0.21 |
| 6 | 140 | 360 | 0.42 |

** Trolox standard

The results are shown in Table 3.

TABLE 3

| | | | Comparative |
|---|---|---|---|
| | Example 1 | Example 2 | Example 1 |
| 1 | 10.99 | 10.76 | 7.74 |
| 2 | 10.42 | 10.38 | 8.01 |
| 3 | 10.98 | 10.69 | 7.81 |
| Ave | 10.87 | 10.61 | 7.85 |
| SD | 0.190 | 0.200 | 0.14 |

Radical scavenging ability measurement result (unit: %)

As a result of the ABTS assay, Examples 1 and 2 respectively showed, at 5% concentration, the average radical scavenging activity of 10.61% and 10.87%, which are statistically significant values compared to the negative control.

### <Example 5> Phototoxicity Test

This test was to evaluate phototoxicity after treating cells of BALB/3T3 clone A31 with a sample in the in vitro 3T3 NRU phototoxicity test. The phototoxicity was identified by evaluating cell viability, IC₅₀ according to the presence or absence of light irradiation, and phototoxicity according to concentration response [photo irritation factor (PIF), and mean photo effect (MPE)]. Evaluation results are as follows.

The concentration of the test substance in this test was set to be 1000, 500, 250, 125, 62.5, 31.25, 15.63 and 7.81 μg/ml concentrations for both ultraviolet (UV) irradiation and non-irradiation through a dose setting test. In addition, as a negative control group, PBS diluted in DMEM was used.

As a result of calculating the IC₅₀, which is the concentration value at which the cell viability decreases to 50% according to the presence or absence of light irradiation, it was found that in the test substance group, the IC₅₀ value was not calculated during UV irradiation and non-irradiation; and in a positive control material group, when UV was not irradiated, the IC₅₀ was 28.043 μg/ml, and when UV was irradiated, the IC₅₀ was 1.650 μg/ml.

As a result of evaluating the phototoxicity according to the concentration response in the test substance group, PIF was less than 2 and MPE was less than 0.1 (PIF: Not applicable, and MPE: −0.094), and in the positive control material group, PIF was more than 6 and MPE was 0.15 or more (PIF: 17.1444, MPE: 0.459).

In the positive control material group, when UV was irradiated, the ICs ranged from 0.1 μg/ml to 2.0 μg/ml, and when UV was not irradiated, the ICs ranged from 7.0 μg/ml to 90.0 μg/ml and PIF was 6 or more. From these results, it was confirmed that the test substance was a phototoxic material.

In all groups, the cell viability of the negative control material under UV irradiation with respect to the non-UV irradiation was 80% or more, and the average absorbance value of the negative control material was 0.4 or more. In this test, it was judged that the test was properly conducted because the conditions for establishing the test were satisfied.

From the above results, it was determined that the result obtained by evaluating the phototoxicity of polysaccharides using the BALB/3T3 clone A31 cell line, is “No phototoxicity”.

### <Example 5-1> Test Method

(1) Preparation and Analysis of Test Substances

(1-1) Preparation of Test Substances

Each of test substances was dissolved in PBS and diluted in steps at azeotrope 2. Immediately before treatment, each test substance was diluted 10 times with DMEM to prepare a final concentration thereof. As the test substances, the functional polysaccharide of Example 1 was used.

(1-2) Preparation of Positive Control Material

CPZ, a positive control material, was dissolved in ethanol and diluted in steps at azeotrope 2, and then immediately before treatment with the test substance, each concentration was diluted 100 times with DMEM to obtain the final concentration of the positive control material.

(1-3) Analysis of Test Substance

In consultation with the test sponsor, the concentration, stability and homogeneity of the test substance and test substance preparation were not separately analyzed.

(2) Preparation of Cell Line

(2-1) Cell Line

Cell line: BALB/3T3 clone A31

Source: American Type Culture Collection

Lot No.: 62485414

(2-2) Reasons for Cell Line Selection

The BALB/3T3 clone A31 cell used in this test is a cell that is widely used to evaluate phototoxicity. Accordingly, there are many test data accumulated to be compared, which may lead to ease of interpretation of test results.

(2-3) Preservation

Cells were suspended in a medium to which DMSO was added at a final ratio of 10% (v/v), dispensed by about 1 ml, and frozen in Oct. 19, 2015, transferred to a liquid nitrogen storage container, and stored. In the test, the cells were thawed and incubated before or during the test period, and were used in common with other tests.

(2-4) Culture Conditions

Temperature: (37±1)° C.

CO₂ concentration: 5%

Humidity: Humidification conditions

Incubator: CO₂ cell incubator (NUAIRE, NU-4850E)

(2-5) Confirmation of Cell Line Type and Cell Contamination

For the cells used, the cell morphology was visually observed and the contamination of mycoplasma was confirmed on Mar. 29, 2019 by using the Hoechst Stain Kit (MPBIOMEDICALS, Japan). (Observation of morphology: appropriate, confirmation of contamination: negative)

(2-6) UV Sensitivity Test of Cells

For the cells used, the UV sensitivity of the cells according to the amount of light was identified on May 15, 2019 using an ultraviolet irradiator.

(3) Cell Culture Solution

DMEM medium was purchased and prepared before or after the start of the test and used in common with other tests.

Complete medium

DMEM(Dulbecco's modified eagle's medium): NBCS(Newborn Calf Serum): Penicillin Streptomycin=89:10:1

Serum free medium

DMEM: Penicillin Streptomycin=99:1

(4) Reagent

Reagents were used in the following composition.

Neutral red medium

Serum free medium: Neutral red solution (0.33%)=65:1

Neutral red extract

Sterile distilled water: Ethanol (99.9%) Absolute: Acetic acid=49:50:1

(5) UV Irradiation

(5-1) UV Irradiation Device

Model Name: BIO-SUN

Serial number: 15-101159

Distance between light source and test system: minimum 23 cm, maximum 26 cm

(5-2) Light Source and Amount of Light

UVA (315-400 nm) causing a phototoxic reaction by activating chemical materials without toxicity to cells was irradiated, and the amount of light was 5 J/cm².

(5-3) Exposure Time

For the amount of light (5 J/cm²), the exposure time was calculated by applying the light intensity measured 1 minute after the start of UV irradiation.

The light intensity was maintained at 1.7±0.5 Mw/cm².

$$t ( min ) \frac{amount of light ( \frac{J}{{cm}_{2}} ) \times 1000}{i n t e nsity of light ( \frac{mW}{{cm}_{7}} ) \times 60} , ( 1 J = 1 Wsec )$$

(6) Dose Setting Test

Test substances were set at 1000, 500, 250, 125, 62.5, 31.25, 15.63, ad 7.81 μg/ml concentrations for both UV irradiation and non-irradiation. Cell viability was calculated to identify cytotoxicity, and PIF and MPE values were not calculated.

| | |
|---|---|
| Case in which cytotoxicity did not | Main test was performed at the |
| occur, and precipitation did not | same concentration as the dose |
| occur when treatment with test | setting test |
| material is finished | |
| Case in which cytotoxicity did not | Minimum dose at which precipitation |
| occur, and precipitation occurred | is formed, was used as the maximum |
| when treatment with test material | dose of the main test, and 8 doses |
| is finished | were set at least one azeotrope 2 |
| Case in which cytotoxicity | IC50, which is a dose corresponding |
| occurred | to about 50% inhibition of cell |
| | proliferation, was used as a medium |
| | dose, based on which 8 |
| | concentrations of main test are set. |

<Dose Setting Test References>

(7) Main Test

(7-1) Composition of Test Group

| Group | | Treatment concentration (μg/ml) |
|---|---|---|
| G1 | UV (−) | 1000, 500, 250, 125, 62.5, 31.25, 15.63, 7.81, 0* |
| (test | UV (+) | 1000, 500, 250, 125, 62.5, 31.25, 15.63, 7.81, 0* |
| material) | | |
| G2 | UV (−) | 200, 100, 50, 25, 12.5, 6.25, 3.13, 1.56, 0* |
| (positive | UV (+) | 5, 2.5, 1.25, 0.63, 0.31, 0.16, 0.08, 0.04, 0* |
| control | | |
| material | | |

*G1 and G2 negative controls were PBS diluted 10 times in DMEM. As for the concentration of G1, in the dose setting test, IC₅₀ was not calculated, and the precipitation of the test substance was not confirmed. Accordingly, the main test was conducted at the same concentration as the dose setting test.

(7-2) Test Proceeding

Cell dispensing: 1 day

100 μl of cell suspension (1×10⁴ cells/well) and medium were dispensed into two 96 well plates per group, and the cells were cultured for 24 hours in a cell incubator at 37° C. and 5% CO².

test substance treatment: 2 days

After removing the medium, the cells were washed once with 150 μl of warm PBS. After treatment with 100 μl of the preparation (test substance and positive control material) and the negative control material, the cells were incubated for 1 hour in a cell incubator at 37° C. and 5% CO².

UV irradiation: 2 days

The UV irradiation plate was irradiated with UVA at a light intensity of 5 J/cm², and while UV was irradiated, the non-irradiated plate was shielded with aluminum foil and left at room temperature. After removing the solution, washing was performed twice with 150 μl of warm PBS. 100 μl of the medium was dispensed and the cells were cultured for 20±2 hours in a cell incubator at 37° C. and 5% CO₂.

Neutral red extraction: 3 days

The growth and morphology of the cells were observed using a microscope, the medium was removed therefrom, and then the cells were washed once with 150 μl of warm PBS. After dispensing 100 μl of neutral red medium, the cells were incubated for 3 hours in a cell incubator at 37° C. and 5% CO₂. After removing the neutral red medium, the cells were washed once with 150 μl of warm PBS. 150 μl of neutral red extract was dispensed, and the plate was stirred for 10 minutes while light was shielded.

Absorbance measurement: 3 days

Absorbance was measured at a wavelength of 540 nm by using a multi-channel microplate reader.

(8) Test Establishment Conditions

The cell viability of the negative control group when UV was irradiated was 80% or more of the cell viability thereof when UV was not irradiated.

The average absorbance value of the negative control group was 0.4 or more.

The IC₅₀ of the positive control material (CPZ) was 0.1 μg/ml-2.0 μg/ml when UV was irradiated, and 7.0 μg/ml-90.0 μg/ml when UV was not irradiated, and the PIF value was 6 or more.

(9) Evaluation and Judgment of Results

(9-1) Evaluation

IC₅₀ was evaluated according to the presence or absence of light irradiation.

Phototoxicity was evaluated by PIF and MPE according to the concentration response of each material.

(9-2) Judgment

Phototoxicity was judged by calculating PIF and MPE values using IC₅₀ according to the presence or absence of light irradiation.

| PIF | MPE | |
|---|---|---|
| (Photo irritation faster) | (Mean photo effect) | Evaluation results |
| PIF < 2 | MPE < 0.1 | No phototoxicity |
| 2 < PIF < 5 | 01. < MPE < 0.15 | Probable phototoxicity |
| 5 < PIF | 0.15 < MPE | Phototoxicity |

(10) Statistics

PIF and MPE values were calculated from the measured absorbance values by using the software (Phototox 2.6, ZEBET at the BFR, Berlin Germany), and no statistical method was performed.

### <Example 5-2> Results

(1) Comparison of IC₅₀ According to the Presence or Absence of Light Irradiation

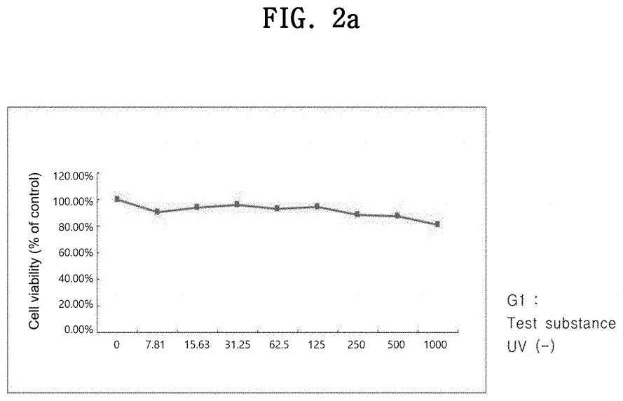

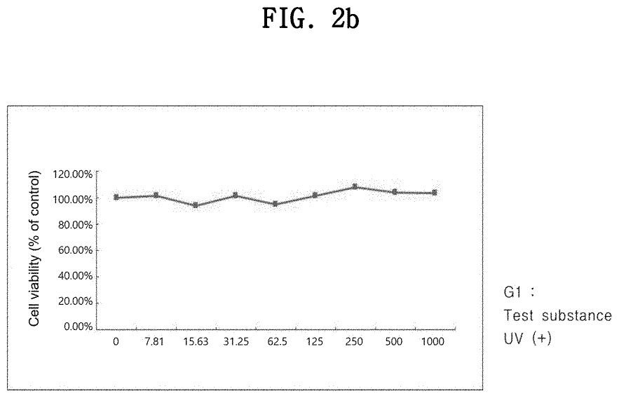

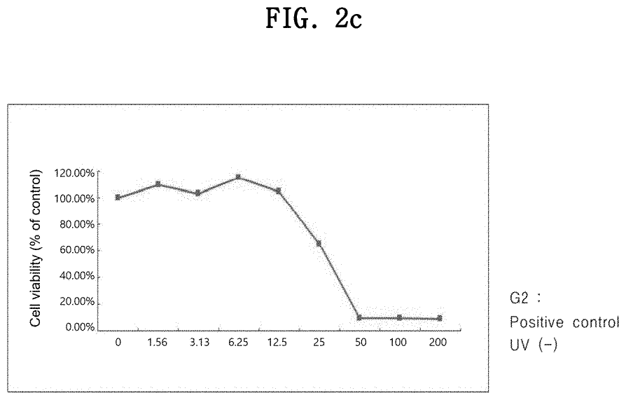

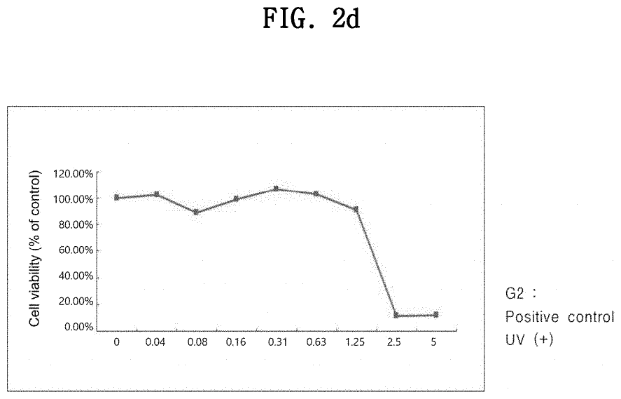

As a result of calculating the ICs, which is the concentration value at which the cell viability decreases to 50% with or without light irradiation, it was found that, in the test substance group, the ICs value was not calculated at the time of UV irradiation and UV non-irradiation, and in the positive control material group, the IC₅₀ was 28.043 μg/ml when UV was irradiated, and the IC₅₀ was 1.650 μg/ml when UV was not irradiated. (FIG. 2a to 2d)

| | | Concentration | OD₅₄₀^{a)} | | Cell viability | IC₅₀^{b)} |
|---|---|---|---|---|---|---|
| Group | | (μg/mL) | Mean | S.D. | (% of NC) | (μg/mL) |
| G1 (TS) | UV (−) | 0 | 0.570 | 0.100 | 100.00 | — |
| | | 7.81 | 0.516 | 0.072 | 90.40 | |
| | | 15.63 | 0.536 | 0.085 | 93.88 | |
| | | 31.25 | 0.547 | 0.050 | 95.84 | |
| | | 62.5 | 0.531 | 0.042 | 93.06 | |
| | | 125 | 0.539 | 0.039 | 94.46 | |
| | | 250 | 0.504 | 0.067 | 88.39 | |
| | | 500 | 0.499 | 0.026 | 87.54 | |
| | | 1000 | 0.462 | 0.010 | 81.05 | |
| | UV (+) | 0 | 0.531 | 0.043 | 100.00 | — |
| | | 7.81 | 0.539 | 0.053 | 101.49 | |
| | | 15.63 | 0.499 | 0.041 | 93.96 | |
| | | 31.25 | 0.537 | 0.045 | 101.24 | |
| | | 62.5 | 0.505 | 0.023 | 95.12 | |
| | | 125 | 0.539 | 0.031 | 101.55 | |
| | | 250 | 0.574 | 0.090 | 108.12 | |
| | | 500 | 0.552 | 0.058 | 104.04 | |
| | | 1000 | 0.549 | 0.059 | 103.47 | |
| G2 (PC) | UV (−) | 0 | 0.537 | 0.033 | 100.00 | 28.043 |
| | | 1.56 | 0.589 | 0.081 | 109.65 | |
| | | 3.13 | 0.554 | 0.032 | 103.07 | |
| | | 6.25 | 0.617 | 0.090 | 114.87 | |
| | | 12.5 | 0.562 | 0.044 | 104.59 | |
| | | 25 | 0.352 | 0.271 | 65.55 | |
| | | 50 | 0.051 | 0.002 | 9.50 | |
| | | 100 | 0.050 | 0.001 | 9.37 | |
| | | 200 | 0.050 | 0.001 | 9.31 | |
| | UV (+) | 0 | 0.514 | 0.033 | 100.00 | 1.650 |
| | | 0.04 | 0.528 | 0.039 | 102.67 | |
| | | 0.08 | 0.456 | 0.044 | 89.09 | |
| | | 0.16 | 0.508 | 0.035 | 98.75 | |
| | | 0.31 | 0.549 | 0.071 | 106.73 | |
| | | 0.63 | 0.530 | 0.052 | 103.10 | |
| | | 1.25 | 0.467 | 0.050 | 90.78 | |
| | | 2.5 | 0.059 | 0.012 | 11.51 | |
| | | 5 | 0.063 | 0.011 | 12.32 | |

※ TS: Test substance, PC: Positive control, NC: Negative control
^{a)}OD₅₄₀: Mean out by 6 wells (Negative control: 12 wells)
^{b)}IC₅₀: 50% inhibitory concentration
S.D.: Standard deviation

(2) Phototoxicity (PIF and MPE) Evaluation According to Concentration Reaction

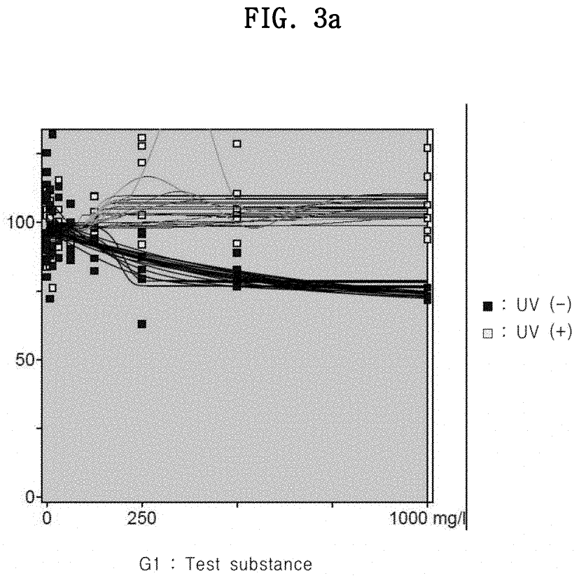

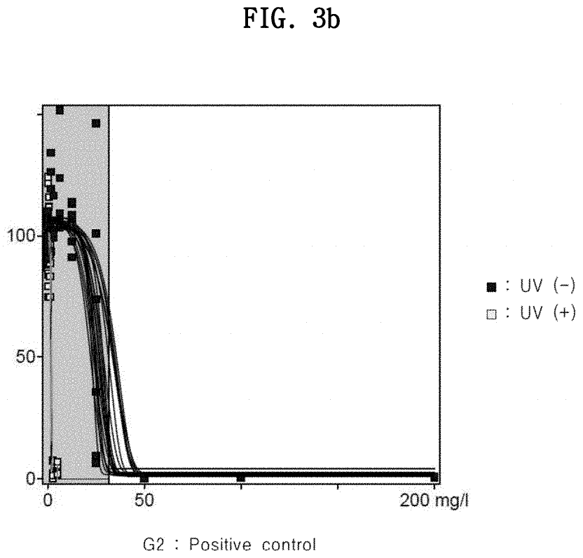

As a result of calculating the PIF and MPE values by using IC₅₀ according to the presence or absence of light irradiation, in the test substance group, the PIF value was not calculated and the MPE value was −0.094, and in the positive control material group, the PIF value was 17.144, and the MPE value was 0.459 (FIGS. 3a and 3b)

| Group | PIF (Photo imitation factor) | MPE (Mean photo effect) |
|---|---|---|
| G1 | — | −0.094 |
| (TS) | | |
| G2 | 17.144 | 0.459 |
| (PC) | | |

※ TS: Test substance, PC: Positive control

(3) Comprehensive Evaluation

To evaluate the phototoxicity of functional polysaccharides against cells of BALB/3T3 clone A31, cell viability, IC₅₀ according to the presence or absence of light irradiation, and phototoxicity according to concentration response (PIF and MPE) were calculated as follows.

As a result of calculating the IC₅₀, which is the concentration value at which the cell viability decreases to 50% with or without light irradiation, it was found that, in the test substance group, the IC₅₀ value was not calculated at the time of UV irradiation and UV non-irradiation, and in the positive control material group, the IC₅₀ was 28.043 μg/ml when UV was irradiated, and the IC₅₀ was 1.650 μg/ml when UV was not irradiated. As a result of calculating the PIF and MPE values by using IC₅₀ according to the presence or absence of light irradiation, in the test substance group, the PIF value was not calculated and the MPE value was −0.094, and in the positive control material group, the PIF value was 17.144, and the MPE value was 0.459.

In the positive control material group, when UV was irradiated, the ICs ranged from 0.1 μg/ml to 2.0 μg/ml, and when UV was not irradiated, the ICs ranged from 7.0 μg/ml to 90.0 μg/ml and PIF was 6 or more. From these results, it was confirmed that the test substance was a phototoxic material. In all groups, the cell viability of the negative control material under UV irradiation with respect to the non-UV irradiation was 80% or more, and the average absorbance value of the negative control material was 0.4 or more. In this test, it was judged that the test was properly conducted because the conditions for establishing the test were satisfied.

From the above results, it was determined that the result obtained by evaluating the phototoxicity of polysaccharides using the BALB/3T3 clone A31 cell line, is “No phototoxicity”.

### <Example 6> Human Skin Irritation Test

(1) Test Summary

In this study, an experiment was conducted on Korean adult men and women for the primary human skin irritation test of two raw materials provided by the Seowon University Industry-Academic Cooperation Foundation.

After the patch was removed, the judgment was carried out two times, and the average skin response degree was calculated by combining the results of the first and second judgments. The final judgment was made based on the average skin response degree.

(2) Test Substance

test substance

Name of material: CT16-SA25 (Example 1), CT16-SA24 (Example 2)

Date of manufacture: May 15, 2018

Product type (appearance and characteristics): liquid form

Storage conditions: Stored between 5° C. to 25° C., without exposure to high temperature and direct sunlight

(3) Subject

Information on the subjects who participated in this human application test is shown in Table 6.

Table 6. Subject information

(4) Test Method

(4-1) Test Method

A raw material was attached in the form of a closed patch on the subject's back (excluding the spine) for 24 hours, and then the patch was removed.

After removing the patch from the subject's back (excluding the spine), the site was marked, and then, impurities were removed from the back (excluding the spine) using the tissue for experimental use (Kimtech). The first judgment evaluation was carried out 30 minutes after the patch was removed, and the second judgment evaluation was conducted 24 hours later. The average skin response degree was evaluated by integrating two judgment evaluations for the product, and the final judgment evaluation was conducted based thereon.

All judgments and evaluations were conducted after the subject had stabilized the skin for at least 30 minutes under constant temperature and humidity conditions without the movement of air and direct sunlight.

(4-2) Test Site Selection and Patch Attachment

The back (excluding the spine) was selected as the patch attachment site.

The product was cut into 8×8 mm and placed on a patch, which was then attached to the back. For the negative control group, distilled water was dropped, and the space of the patch in which nothing was dropped was set as blank.

10 Ultra Chamber (Chemotechnique Diagnostics, Sweden) was used as a patch to evaluate skin irritation. IQ Ultra Chamber has 10 inert polyethylene chambers arranged on a rectangular-shaped non-irritating adhesive tape with a width of 5 mm and a length of 118 mm, and each chamber has the volume of up to 32 μl and the area of 8×8 mm. The spacings between neighboring chambers are designated as 12 mm between columns and 15 mm between lines.

(4-3) Judgment of Skin Irritation

The patch was attached for 24 hours and then removed. After waiting for the disappearance of transient erythema due to removal, the patch was removed. 30 minutes after the removal, the first judgment was conducted.

As the irritation criterion, the reading criteria of the International Contact Dermatitis Research Group (ICDRG) as shown in Table 7 were applied and a measurement evaluation method devised by Frosch & Kligman was used.

| Response degree | Type of skin symptoms or signs |
|---|---|
| 1 (+) | Slight erythema, either spotty or diffuse |
| 2 (++) | Moderate uniform erythema |
| 3 (+++) | Intense erythema with edema |
| 4 (++++) | Intense erythema with edema & vesicles |
| Negative (−) | negative response |

Criteria for Determining Irritation

(4-4) Data Analysis

The average skin response degree (mean score) was calculated according to the calculation formula of the following formula.

$$Average skin response degree = \frac{\sum ( Each score : sum of observation values during judge )}{Maximum mean score 4 \times total number of paticipants \times 2} \times 100$$

As for the criteria for skin response degree to the product, the final judgment was conducted according to the criteria shown in Table 8 below according to the mean score value.

| Evaluation results (Grade) | Mean score |
|---|---|
| No irritation | 0.0-0.9 |
| Weak irritation | 1.0-2.9 |
| Middle irritation | 3.0-4.9 |
| Strong irritation | 5.0- |

Final Judgment Criteria

(5) Test Results

(5-1) Analysis of Skin Irritation Evaluation Results

The judgment results are as follows.

| | Number of | | | | |
|---|---|---|---|---|---|
| | partic- | | | Average | |
| | ipants | | | skin | |
| | having | Response degree | | response | Evaluation |
| Product No. | responses | 30 min | 24 hr | degree | results |
| CT16-SA 24 | 0 | 0 | 0 | 0 | No irritation |
| CT16-SA 25 | 0 | 0 | 0 | 0 | No irritation |
| Distilled water | 0 | 0 | 0 | 0 | No irritation |
| (Vehicle, | | | | | |
| negative | | | | | |
| control group) | | | | | |
| Blank | 0 | 0 | 0 | 0 | No irritation |

*average skin response degree: see the formula described above

(6) Conclusion

This test is the primary skin irritation test for human body, and was conducted in accordance with the regulations of the Ministry of Food and Drug Safety Notification No. 017-4, functional cosmetics review.

A total of 33 subjects were recruited, and a total of 2 dropouts occurred including one as a case of not following the restrictions or obligations of the test, and one as a case where the test manager judges that the subject cannot continue the test. Finally, 31 people completed the test.

Test results showed that the average skin response degree of CT16-SA24 and CT16-SA25 was calculated as 0. That is, all were judged as non-irritating according to the final criteria.

| Product No. | Average skin response degree |
|---|---|
| CT16-SA 24 | No irritation |
| CT16-SA 24 | No irritation |

### <Example 7> Cytotoxicity Test

(1) Summary

A cytotoxicity test (MTT assay) was conducted using CCD-986SK (Fibroblast) cells to evaluate the cytotoxicity of a functional polysaccharide. As the test substance, the functional polysaccharide of Example 1 was used after being diluted.

As a result of the treatment with the functional polysaccharide having concentrations of 0.1%, 0.5%, 1.0%, and 2.0%, a cell viability of more than 70% was observed at the concentration of 2.0% or less.

The results of the cytotoxicity test (MTT assay) using CCD-986SK (Fibroblast) cells showed that the functional polysaccharide had no cytotoxicity at a concentration of 2.0% or less.

(2) Test Method

Cell line: CCD-986SK Source: ATCC CRL-6323TM Lot No.: 60016875

Cells suspended in the cell line-preserved freezing medium, were dispensed at about 1 mL each, frozen on Sep. 16, 2015, and transferred to a liquid nitrogen storage container for storage. For the test, the cells were thawed and incubated before or during the test period before use.

[symbol] Culture conditions

Temperature: (37±1°) C, CO₂ concentration: 5%, humidity: humidification conditions, and incubator: CO₂ cell incubator

Cell culture solution

Complete medium

Dulbecco's modified Eagle's medium (DMEM): 445 ml

Fetal Bovine Serum (FBS): 50 ml

Penicillin-Streptomycin: 5 ml

Total volume: 500 ml

Serum-free medium

Dulbecco's modified Eagle's medium (DMEM): 99 ml

Penicillin-Streptomycin: 1 ml

Total volume: 100 ml

Cytotoxicity test

The concentration of the test substance in a group was diluted to 4 concentrations (0.1%, 0.5%, 1.0%, and 2.0%) and the test was carried out as follows.

G1: negative control

G2: 0.1% functional polysaccharide

G3: 0.5% functional polysaccharide

G4: 1.0% functional polysaccharide

G5: 2.0% functional polysaccharide

Cells were dispensed into a 96 well plate at 3×10⁴ (100 uL/well) and incubated for 24 hours in an incubator having the temperature of 37° C. and the condition of 5% CO₂.

Test substance treatment: the medium was replaced with a serum-free medium containing a test substance which had been diluted at various concentrations and the cells were incubated for 24 hours in an incubator having the temperature of 37° C. and the condition of 5% CO₂.

MTT assay: after washing twice with PBS, MTT solution (0.5 mg/mL, serum-free medium) was dispensed at 200 μL/well, and the cells were incubated for 4 hours in an incubator having the temperature of 37° C. and the condition of 5% CO₂.

Measurement of absorbance: after the incubation was finished, the medium was removed, a DMSO solution was added at 200 μL/well, an extraction was performed for 20-30 minutes in a plate shaker, and the absorbance was measured using ELISA at a wavelength of 570 nm.

Result processing: when the cell viability was decreased to less than 70% of the sample-free group, it is judged that there is potential cytotoxicity and the cell viability is calculated according to the following equation.

$$Cell viability ( \% ) = \frac{O . D . of sample addition group at 570 nm}{O . D . of sample non - addition group at 570 nm} \times 100$$

Statistical processing: whether experiment results have significance, was evaluated by performing t-test (paired t-test, two-sided verification) using the SPSS (Ver. 19) statistical program.

(3) Results

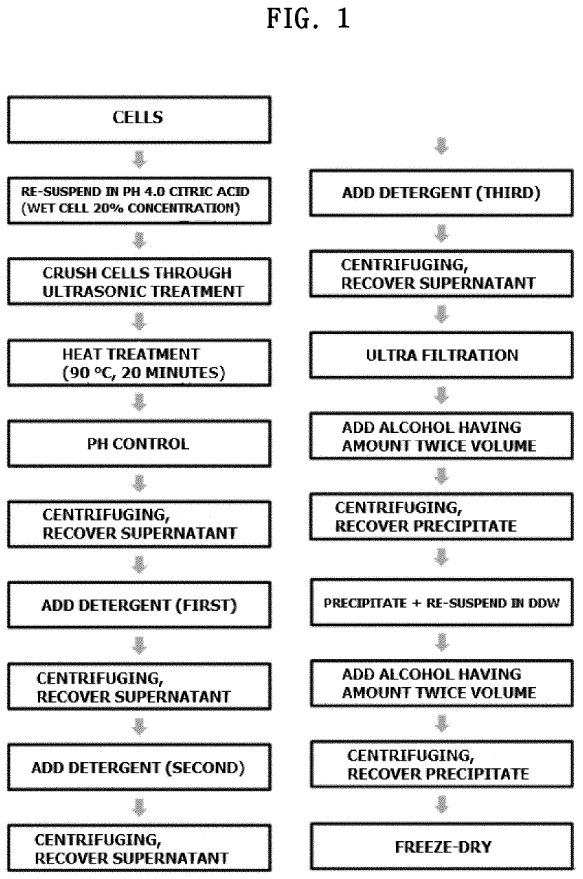

Cytotoxicity evaluation (Table 1, FIG. 1, Appendix 1): when treated with 0.1%, 0.5%, 1.0%, and 2.0% functional polysaccharides, the cell viability was 105.96±29.69%, 86.66±14.42%, 126.61±16.46%, and 102.54±21.75%, respectively.

(4) Discussion and Conclusion

To evaluate the cytotoxicity of a functional polysaccharide, a cytotoxicity test (MTT assay) of a functional polysaccharide was performed using CCD-986SK (Fibroblast) cells and a diluted solution of functional polysaccharide.

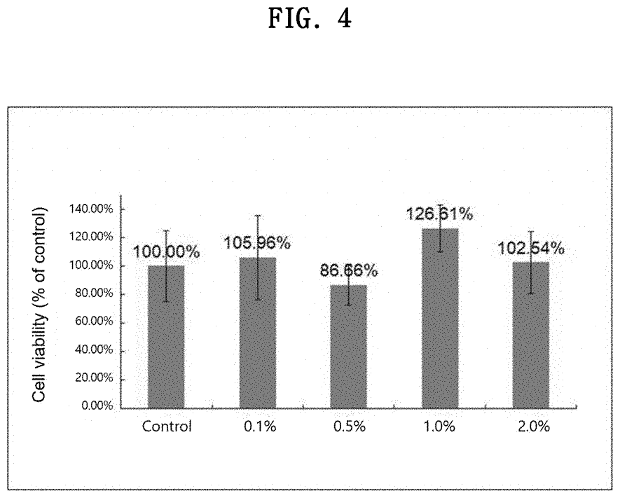

As shown in FIG. 4, the results of the cytotoxicity test show that, when treated with 0.1%, 0.5%, 1.0%, and 2.0% functional polysaccharides, the cell viability was 105.96±29.69%, 86.66±14.42%, 126.61±16.46%, and 102.54±21.75%, respectively.

Functional polysaccharide: at a concentration of 2.0% or less, a cell viability was 70% or more.

When the cell viability is decreased to less than 70% of the sample-free group, it is judged that there is potential cytotoxicity. Accordingly, the functional polysaccharide is considered to have no cytotoxicity at the concentration of 2.0% or less. From the above results, it can be seen that the functional polysaccharide used in the cell cytotoxicity test (MTT assay) using CCD-986SK (Fibroblast) has no cytotoxicity at a concentration of 2.0% or less.

| | | | | OD Value | |
|---|---|---|---|---|---|
| Group | Substance | Dose | | (570 nm) | % of control |
| G1 | Control | — | Mean | 2.41 | 100.00 |
| | | | S.D. | 0.61 | 25.21 |
| | | | N | 9 | 9 |
| G2 | Glycogen | 0.1% | Mean | 2.56 | 105.96 |
| | | | S.D | 0.72 | 29.69 |
| | | | N | 9 | 9 |
| G3 | | 0.5% | Mean | 2.09 | 86.66 |
| | | | S.D. | 0.35 | 14.42 |
| | | | N | 9 | 9 |
| G4 | | 1.0% | Mean | 3.06 | 126.61 |
| | | | S.D. | 0.40 | 16.46 |
| | | | N | 9 | 9 |
| G5 | | 2.0% | Mean | 2.48 | 102.54 |
| | | | S.D | 0.53 | 21.75 |
| | | | N | 9 | 9 |

** Evaluation results of cytotoxicity according to dilution concentration

### <Example 8> Evaluation of Skin Moisturizing Elasticity Enhancing Effect (Cosmetics)

(1) Summary

All 12 subjects who ended this test were women, and their average age was 45.4 years. The selected subjects had no specific skin symptoms and had no history of disease or drug use that could affect the test.

Regarding the effect of improving skin moisturization, the moisture content was increased statistically significantly (P<0.05) after 2 and 4 weeks of use compared to before use of the product.

Regarding skin elasticity, the R7 (biological elasticity) value was statistically significantly increased (P<0.05) after 4 weeks of use compared to before use of the product.

Subjective questionnaire evaluation on test subjects: after using the test substance, all items of efficacy evaluation were evaluated as ‘normal’ or higher.

Evaluation of adverse reactions by a dermatologist: during the period of use of the test product, there were no reports of specific adverse skin reactions in the test subjects, and no abnormal findings were observed even on the physical examination by a dermatologist.

(2) Test Substance

Test substance

Name of material:

### Cosmetic Cream a Containing Polysaccharide of Comparative Example 1

### Cosmetic Cream B Containing the Polysaccharide of Example 1

Product type (appearance and characteristics): white creamy form

Storage conditions: room temperature [(1° C.-30° C.)]

Cosmetic cream ingredients: as follows

| No. | Source name | INCI Name | Amount (wt %) |
|---|---|---|---|
| 1 | LANETTE-O | CETEARYL ALCOHOL | 0.50-1.50 |
| 2 | ARLACEL 165 VEG | GLYCERYL STEARATE(AND)PEG-100 STEARATE | 0.10-1.00 |
| 3 | GMS-205 | GLYCERYL STEARATE | 0.10-1.00 |
| 4 | BEES WAX | BEESWAX | 0.10-1.50 |
| 5 | SQUALANE | SQUALANE | 1.00-5.00 |
| 6 | PALMESTER 3595 | APRYLIC/CAPRIC TRIGLYCERIDE | 1.00-5.00 |
| 7 | CEH | CETYL ETHYLHEXANOATE | 1.00-5.00 |
| 8 | SILICONEOIL 200F/100CS | DIMETHICONE | 0.10-1.00 |
| 9 | TEGO CARE 450 | POLYGLYCERYL-3 METHYLGLUCOSE DISTEARATE | 0.50-1.50 |
| 10 | Distilled water 1 | D.I.WATER | To100 |
| 11 | EGY-820 | ETHYL HEXANDIOLGLYCERYL CAPRYLATE | 0.10-1.00 |
| 12 | DPG-FC | DIPROPYLENE GLYCOL | 1.00-3.00 |
| 13 | ELOGLYN R980 | GLYCERIN | 1.00-5.00 |
| 14 | AMINOCOAT | BETAINE | 0.10-1.00 |
| | (Natural Extract BP20) | | |
| 15 | KELTROL F | XANTHAN GUM | 0.01-0.20 |
| 16 | CARBOPOL 941 | CARBOMER | 0.09-0.20 |
| 17 | TRIS AMINO ULTRA PC | TROMETAMINE | 0.50-2.00 |
| | (10% Soln.) | | |
| 18 | Glycogen(7%) | Water/Glycogen | 5.00 |
| 19 | Jinkyeong Complex | Pueraria Thunbergiana Root Extract | 0.10-2.00 |
| | | Glycyrrhiza Glabra (Licorice) Root Extract | |
| | | Paeonia Lactiflora Root Extract | |
| | | Poncirus Trifoliata Fruit Extract | |
| | | Corydalis Turtschaninovii Root Extract | |
| | | Cnidium Officinale Root Extract | |
| | | Saccharomyces Ferment Filtrate | |
| 20 | PREFUME | FRAGRANCE | qs |

| | |
|---|---|
| Total | 100.00 |

(3) Test subjects

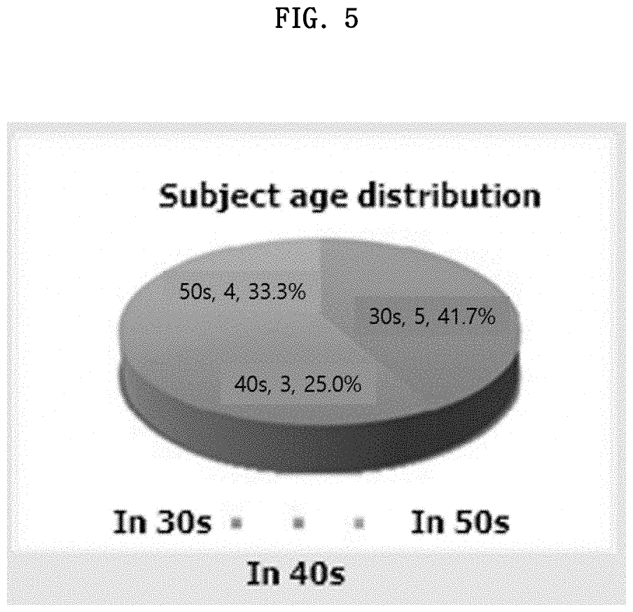

All 12 people recruited for the test were selected as final subjects. In the first place, the target number of subjects was 22 based on the number of test subjects suggested by the Ministry of Food and Drug Safety ‘test method guideline for demonstration of cosmetics labeling advertisement (Guideline for Complainants)’, but at the request of the sponsor, the number of subjects was adjusted to be 12. The test director or the test manager who was delegated by the study director fully informed the test subjects of all the information about the test, and the test subjects filled out the consent form at their will and participated in the test. (FIG. 5)

| Age bracket | Number | Percentage point |
|---|---|---|
| In 30s | 5 | 41.7 |
| In 40s | 3 | 25.0 |
| In 50s | 4 | 33.3 |

** Age distribution of test subjects

(4) Conclusion

A human application test was conducted on the final 12 test subjects to identify the skin moisturizing and skin elasticity improvement effects of the creams containing the polysaccharides of Example 1 and Comparative Example 1.

This test was conducted on 12 healthy adult women aged 30 to 60 years old, and the subjects were guided to use the test product for 4 weeks. The results before using the test product and 2 and 4 weeks after the use of the test product are as follows.

All 12 subjects who ended this test were women, and their average age was 45.4 years. The selected subjects had no specific skin symptoms and had no history of disease or drug use that could affect the test.

| | | | Percentage |
|---|---|---|---|
| Questions | | Average | point (%) |
| Skin type | Dry | 5 | 41.6 |
| | Oily | 0 | 0.0 |
| | Normal | 2 | 16.7 |
| | Combination (Normal to oily) | 2 | 16.7 |
| | Combination (Normal to dry) | 3 | 25.0 |
| Sum | | 12 | 100.0 |

N: Number of Subject

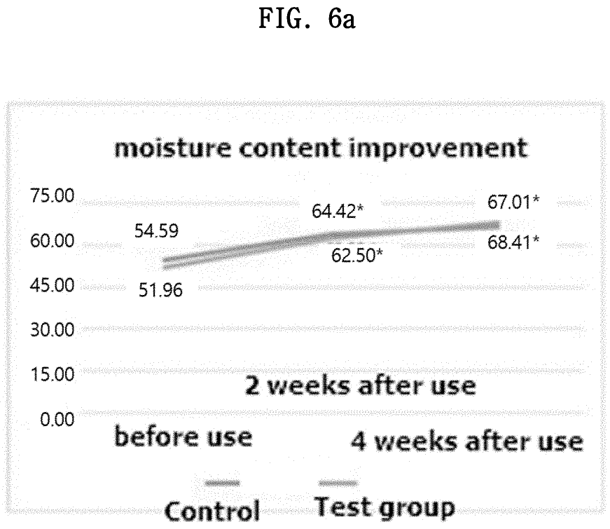

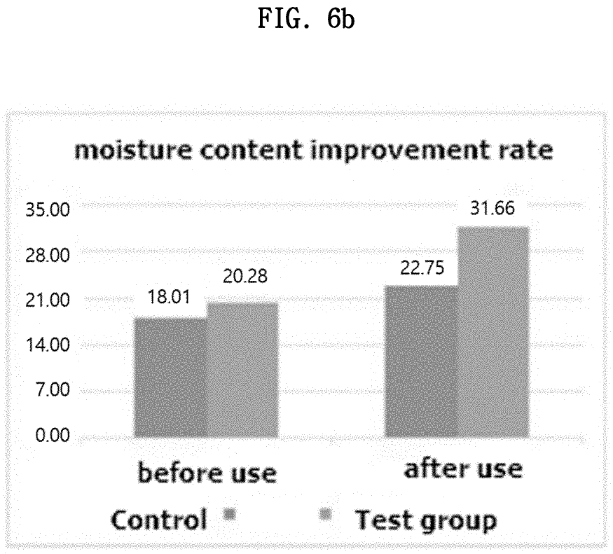

As a result of confirming skin moisturization using a Corneometer, Cream B (containing the polysaccharide of Example 1) showed improvement rates of 20.28% and 31.66%, respectively, after 2 and 4 weeks of use compared to before use, and the skin moisturization rate was statistically significantly increased (P<0.05). Cream A (containing the polysaccharide of Comparative Preparation Example 1) showed improvement rates of 18.01% and 22.75%, respectively, after 2 and 4 weeks of use compared to before use, and the skin moisturization rate was statistically significantly increased (P<0.05). The test product showed a significant difference (P<0.05) after 4 weeks of use compared to the control product. (FIGS. 6a and 6b)

(Unit: A.U., N-12)

| | | before | after | after |
|---|---|---|---|---|
| | | use | 2 weeks | 4 weeks |
| Right cheek | Mean | 54.59 | 64.42* | 67.01* |
| site (test | S.D. | 7.62 | 7.91 | 8.86 |

| | | | |
|---|---|---|---|
| product A) | Improvement rate (%) | 18.01 | 22.75 |

| | | | | |
|---|---|---|---|---|
| [control group] | | | | |
| Left cheek | Mean | 51.96 | 62.50* | 68.41* |
| site (test | S.D. | 7.17 | 10.58 | 8.49 |

| | | | |
|---|---|---|---|
| product B) | Improvement rate (%) | 20.28 | 31.66 |

[test group]

N: Number of Subject,
S.D.: Standard deviation,
*P < 0.05 by Repeated measures ANOVA,
*(P < 0.05 by Mann-Whitney U test)
There is a statistical significance between two groups

<Results of Skin Moisturizing Effect>

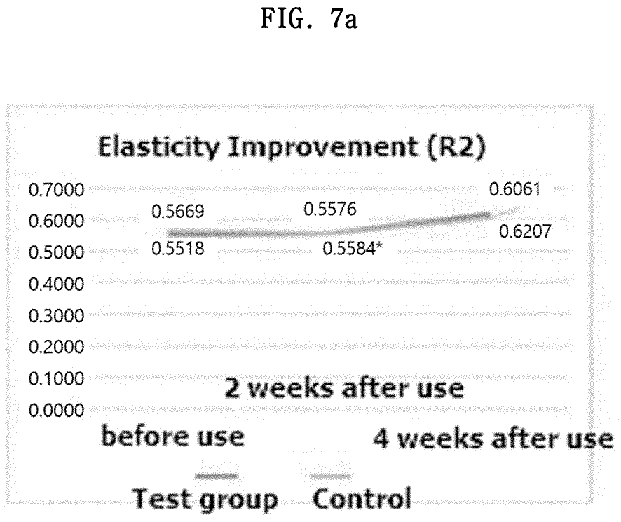

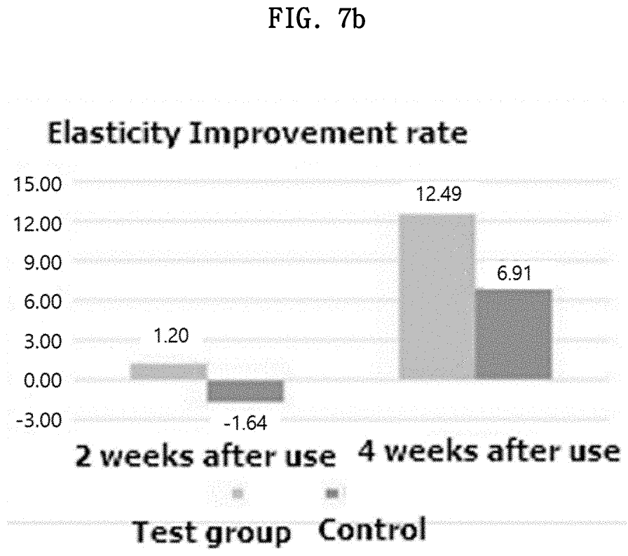

As a result of identifying skin elasticity using a cutometer, the R2 (test product) value of Cream B (test product) showed improvement rates of 1.20% and 12.49%, respectively, after 2 and 4 weeks of use compared to before use. The R5 (net elasticity) value was decreased by −8.1% and −3.33%, respectively, after 2 weeks and 4 weeks of use compared to before use. In other words, the test substance showed no improvement. The R7 (biological elasticity) value was changed by −2.370% and 7.82%, respectively, after 2 and 4 weeks of use compared to before use, and 4 weeks after use, the R7 value was statistically significantly increased (P<0.05). The R2 (gross elasticity) value of Cream A (control product) was changed by −1.64% and 6.91%, respectively, after 2 and 4 weeks of use compared to before use. The R5 (net elasticity) value was decreased by −14.47% and −11.6%, respectively, after 2 weeks and 4 weeks of use compared to before use. In other words, the test product showed no improvement. The R7 (biological elasticity) value was decreased by −5.53% and −0.38%, respectively, after 2 weeks and 4 weeks of use compared to before use. In other words, the test substance showed no improvement. (FIGS. 7a and 7b)

| | right cheek site (test | | before | after | after |
|---|---|---|---|---|---|
| | product B) | | use | 2 weeks | 4 weeks |
| | R2 | Mean | 0.5518 | 0.5584 | 0.6207 |
| | (gross elasticity) | S.D. | 0.0824 | 0.0639 | 0.0795 |
| Improvement rate (%) | | | | 1.20 | 12.49 |
| | R5 | Mean | 0.6448 | 0.5926 | 0.6233 |
| | (net elasticity) | S.D. | 0.0544 | 0.0653 | 0.0697 |
| Improvement rate (%) | | | | −8.10 | −3.33 |
| | R7 | Mean | 0.3963 | 0.3869 | 0.4269* |
| | (biological | S.D. | 0.0438 | 0.0401 | 0.0505 |
| | elasticity) | | | | |

| | | |
|---|---|---|
| Improvement rate (%) | −2.37 | 7.72 |

N: Number of Subject,
S.D.: Standard deviation,
*P < 0.05 by Repeated measures ANOVA

$$Improvement rate ( \% ) \frac{meansurement value after use - measurement value before use}{measurement value before use} \times 100$$

Subjective questionnaire evaluation on test subjects: after using the test substance, all items of efficacy evaluation were evaluated as ‘normal’ or higher.

| Questions | Average | Standard Deviation |
|---|---|---|
| The skin seems to be filled with | 3.8 | 0.7 |
| moisture. | | |
| It seems that the skin tension has | 3.8 | 0.7 |
| decreased. | | |
| The skin feels smooth. | 3.2 | 0.7 |
| The skin seems to have the | 3.3 | 0.9 |
| elasticity. | | |
| It seems that the sagging of the skin | 3.5 | 0.5 |
| has decreased. | | |
| After using the product, it helped | 3.7 | 1.0 |
| improve the overall skin. | | |
| I want to use the product | 3.4 | 1.5 |
| continuously | | |

* 1 = Very unlikely 2 = Unlikely 3 = Normal 4 = Likely 5 = Very likely
** Skin condition after using test substance

Evaluation of adverse reactions by a dermatologist: during the test product was used, there were no reports of specific adverse skin reactions in the test subjects, and no abnormal findings were observed even on the physical examination by a dermatologist.

| | None (0) | | Weak (1) | | Moderate (2) | | Strong (3) | |
|---|---|---|---|---|---|---|---|---|
| | Subject | Tester | Subject | Tester | Subject | Tester | Subject | Tester |
| Erythema | 12 | 12 | 0 | 0 | 0 | 0 | 0 | 0 |
| Edema | 12 | 12 | 0 | 0 | 0 | 0 | 0 | 0 |
| Scaling | 12 | 12 | 0 | 0 | 0 | 0 | 0 | 0 |
| Itching | 12 | 12 | 0 | 0 | 0 | 0 | 0 | 0 |
| Stinging | 12 | 12 | 0 | 0 | 0 | 0 | 0 | 0 |
| Burning | 12 | 12 | 0 | 0 | 0 | 0 | 0 | 0 |
| Tightness | 12 | 12 | 0 | 0 | 0 | 0 | 0 | 0 |
| Prickling | 12 | 12 | 0 | 0 | 0 | 0 | 0 | 0 |

N: Number of Subject
** Results of adverse skin response evaluation

Therefore, Cream B (containing the polysaccharide of Example 1) is judged to be a product that helps to moisturize and improve skin elasticity.

1. A method of producing a functional polysaccharide, the method comprising:
preparing a cell containing a polysaccharide;
obtaining an extract by crushing the cell to extract the polysaccharide to the outside of the cell; and
purifying the extract by removing at least one of crude protein, crude fat, and crude powder from the extract.

2. The method of claim 1, wherein, in the preparing the cell containing the polysaccharide, E. coli mutant TBP38 strain or Saccharomyces Cerevisiae CEY1 is cultured to prepare the cell.

3. The method of claim 1, wherein, in the obtaining of an extract by crushing the cell to extract the polysaccharide to the outside of the cell,
the cell is suspended in a citric acid buffer solution having a pH of 3.5 to 4.5, and then crushed using high pressure treatment to extract the functional polysaccharide to the outside of the cell.

4. The method of claim 1, wherein
the purifying comprises:
heat-treating the extract at 80° C. to 100° C. for 5 minutes to 60 minutes;
adding citric acid to adjust a pH to be 3.5 to 4.5 and removing a precipitate;
adding a detergent thereto to separate layers and recovering a supernatant through centrifugation;
filtering the recovered supernatant through an ultrafine filter and concentrating the same to obtain a concentrate;
adding, to the concentrate, alcohol in an amount 1 to 5 times the volume of the concentrate to recover precipitate; and
re-suspending the precipitate in distilled water or deionized water, and then re-adding alcohol in an amount 1 to 5 times the volume of precipitate and recovering the precipitate.

5. The method of claim 1, wherein an amount of each of crude protein, crude fat, and crude powder is 0.5% or less.

6. The method of claim 1, wherein the polysaccharide is glycogen, and the functional polysaccharide has a purity of 99% or more based on a solid content.

7. A functional polysaccharide prepared by the method of claim 1, wherein an amount of each of crude protein, crude fat, and crude powder is 0.5% or less, and 99% or more of the functional polysaccharide is glycogen based on solid content.

8. The functional polysaccharide of claim 7, wherein the functional polysaccharide has an antioxidant activity.

9. The functional polysaccharide of claim 7, wherein at a concentration of 1000 μg/ml or less, the functional polysaccharide does not have phototoxicity to BALB/3T3 clone A31 cell line.

10. A cosmetic composition for skin moisturizing or skin elasticity improvement, the cosmetic composition comprising the functional polysaccharide of claim 7 as an active ingredient.
